(19) United States
(12) Patent Application Publication
(10) Pub. No.: US 2023/0083200 A1
(43) Pub. Date: Mar. 16, 2023

(54) INJECTION DEPTH INDICATION VIA INJECTION NEEDLE ILLUMINATION

(71) Applicant: Welch Allyn, Inc., Skaneateles Falls, NY (US)

(72) Inventor: Allen R. Hart, Knoxville, TN (US)

(21) Appl. No.: 17/943,036

(22) Filed: Sep. 12, 2022

Related U.S. Application Data
(60) Provisional application No. 63/243,672, filed on Sep. 13, 2021.

(51) Int. Cl. A61M 5/46; A61M 5/19

(52) U.S. Cl. CPC A61M 5/46; A61M 5/19; A61M 2205/587; A61M 2210/0612

(57) ABSTRACT

A medical device includes a head having a base and a piercing member fluidly connected to the base. The medical device includes a tube having a proximal end, a distal end, a first central channel extending from the proximal end to distal end, and a longitudinal axis extending substantially centrally through the first central channel, the distal end of the tube being removably attached to the base. The medical device includes a plunger slidably disposed partly within the first central channel of the tube, the plunger defining a second central channel, and the longitudinal axis extending substantially centrally through the second central channel. In some examples, the medical device includes an optic assembly having a light source and a light pipe that is disposed partly within the second central channel and being configured to receive radiation emitted by the light source; and direct the radiation through the piercing member.

### CROSS REFERENCE TO RELATED APPLICATION

This application is a Nonprovisional of, and claims priority to, U.S. Provisional Patent Application No. 63/243,672, filed Sep. 13, 2021, the entire disclosure of which is incorporated herein by reference.

### TECHNICAL FIELD

This application relates generally to medical equipment, and more particularly, to devices and methods associated with subretinal injections. Additionally, this application is directed to directing light through an injection needle to determine if a particular layer of tissue has been pierced.

### BACKGROUND

Age-related Macular Degeneration (“AMD”) is growing increasingly common, especially among people over the age of 55 years old. While no cure for AMD is currently known, certain retinal conditions, including AMD, may be treated via subretinal drug delivery. One method of providing subretinal drug delivery is focused on providing medicine (e.g., such as a drug) to the patient via an injection into the patient's eye. This method requires a physician to position the needle of a syringe at a specific depth, for example between the choroid and retina.

Delivering medicine to the eye via an injection can present many challenges, such as determining when a needle of a syringe has reached a proper depth to effectively deliver the drug to a patient. For instance, when inserting the needle of the syringe through the outer most layer of the eye (e.g., the sclera), a physician may receive a certain amount of resistance (e.g., tactile feedback) such that they can tell when the needle has pierced that through the sclera. However, the next layers of tissue (e.g., the choroid and the retina), provide little to no tactile feedback to the physician, such that determining when the needle has passed through the choroid and/or the retina is extremely difficult. Additionally, the margin for error for inserting the end of the needle between the choroid and retina is extremely small (e.g., less than 1 millimeter). In some areas, there may be a pocket or gap between the choroid and the retina, while in other areas, there is no gap between the two layers. Accordingly, in some injection procedures, physicians risk piercing the retina and injecting medication into the vitreous or other portions the patient's eye where such medication may be ineffective to treat the patient.

Current solutions for treating AMD and monitoring the depth of a needle during subretinal drug delivery include vitrectomy, which requires a patient to undergo surgery in order for a surgeon to remove the vitreous of the patient's eye and replace it with another solution. A second current solution requires a physician to place a port in the outer layers of a patient's eye and then insert an endoscope into the patient's eye so that the physician can view movement of the needle during injection. However, the above-described current solutions are invasive, and thus, increase the risk of further damage to the patient's eye. Additionally, since AMD does not have a standard treatment, the current solutions may not be covered by insurance such that patients may end up paying for treatment out of pocket. Thus, current solutions may also come at a high financial cost (e.g., cost of surgery, hospital stay, missing work, childcare, pet care, etc.), as well as personal cost (e.g., undergoing surgery, risk of infection, time to recover, etc.) to patients.

A third current solution involves the use of a depth gauge on a needle of a syringe to guide a primary care physician to inject medicine into a patient's eye at the correct depth (e.g., between the choroid and retina). The depth gauge may assist the physician with injecting the needle to a predefined depth within the eye (e.g., 5 millimeters). However, this solution ignores the fact that each patient's eyes are unique, and various factors (e.g., age, health, gender, angle of the needle, location of the injection, etc.) can impact how deep the needle will need to be inserted. Thus, stopping the needle of the syringe at the predefined depth for some patients may result in the treatment being ineffective (e.g., such as when the needle pierces the retina).

Accordingly, current solutions do not provide a safe, simple, cost effective way to enable a primary care physician to effectively administer medication to a patient via a subretinal injection.

The various examples of the present disclosure are directed toward overcoming one or more of the deficiencies noted above.

### SUMMARY

In an example of the present disclosure, a medical device comprises a head having a base and a piercing member fluidly connected to the base. The medical device may further comprise a tube having a proximal end, a distal end, a first central channel extending from the proximal end to distal end, and a longitudinal axis extending substantially centrally through the first central channel, the distal end of the tube being removably attached to the base. The medical device may also comprise a plunger slidably disposed at least partly within the first central channel of the tube, the plunger defining a second central channel, and the longitudinal axis extending substantially centrally through the second central channel. The medical device may comprise an optic assembly having a light source and a light pipe, the light pipe being disposed at least partly within the second central channel and being configured to receive radiation emitted by the light source and direct the radiation through the piercing member.

In another example of the present disclosure, a method comprises providing a first tube comprising a proximal end, a distal end, a first central channel extending from the proximal end to the distal end, and a longitudinal axis extending substantially centrally through the first central channel. In some examples, the method may further comprise providing a light pipe comprising a light pipe tube and a second tube, wherein the light pipe tube is disposed at least partly within a second central channel of the second tube. In some examples, the method comprises disposing the light pipe at least partly within the first central channel of the first tube and connecting a housing to the proximal end of the first tube and operably connected to the light pipe to create an optic assembly, the housing defining an interior space and supporting an input device. In some examples, the method comprises disposing a power source, a light source, and a circuit board at least partly within the interior space of the housing, the circuit board being operably connected to the power source, the light source, and the input device, the light source being operable to selectively direct radiation to the light pipe based on an input received via the input device, and the light pipe being configured to direct the radiation to exit the first tube via the distal end of the first tube.

In still another example of the present disclosure, a method comprises inserting a piercing member of a medical device, through a sclera of an eye, to a first depth within the eye. In some examples, the medical device may comprise: a head having a base and the piercing member fluidly attached to the base; a tube having a proximal end, a distal end, a first central channel extending from the proximal end to distal end, and a longitudinal axis extending substantially centrally through the first central channel, the distal end of the tube being removably attached to the base; a plunger slidably disposed at least partly within the first central channel of the tube, the plunger defining a second central channel, and the longitudinal axis extending substantially centrally through the second central channel; and an optic assembly having a light source and a light pipe, the light pipe being disposed at least partly within the second central channel. In some examples, the method may further comprise directing first radiation, through the piercing member, to impinge upon a retina of the eye at a first location. The method may comprise inserting the piercing member of the medical device, through the retina of the eye, to a second depth within the eye. While the piercing member is disposed at the second depth, the method may comprise directing second radiation through the piercing member to impinge upon an innermost area of the eye at a second location, and visually identifying the second radiation impinging upon the innermost area of the eye at the second location. Based on visually identifying the second radiation, the method may comprise causing the piercing member to be disposed at a third depth within the eye and while the piercing member is disposed at the third depth: determining that the second radiation cannot be visually identified as impinging upon the innermost area of the eye at the second location, and based on determining that the second radiation cannot be visually identified as impinging upon the innermost area of the eye, delivering a treatment within the eye via the piercing member.

### BRIEF DESCRIPTION OF THE DRAWINGS

Examples of the present disclosure may comprise one or more of the features recited in the appended claims and/or one or more of the following features or combinations thereof. Additionally, in this specification and drawings, features similar to or the same as features already described may be identified by reference characters or numerals which are the same as or similar to those previously used. Similar elements may be identified by a common reference character or numeral, with suffixes being used to refer to specific occurrences of the element.

FIG. 1 illustrates an example medical device of the present disclosure. In some implementations, components of the example medical device shown in FIG. 1 may be used to deliver medication via subretinal injection.

FIG. 2A illustrates a partially exploded view of the example medical device shown in FIG. 1.

FIG. 2B illustrates an example view of an assembled example medical device.

FIG. 3 illustrates a cross section of a human eye.

FIG. 4 is an example method associated with the example medical device shown in FIG. 1.

FIG. 5 is an example method associated with the example medical device shown in FIG. 1. Similar to FIG. 1, in some implementations, components of the examples illustrated in FIGS. 2-5 may be used to deliver medication via subretinal injection.

### DETAILED DESCRIPTION

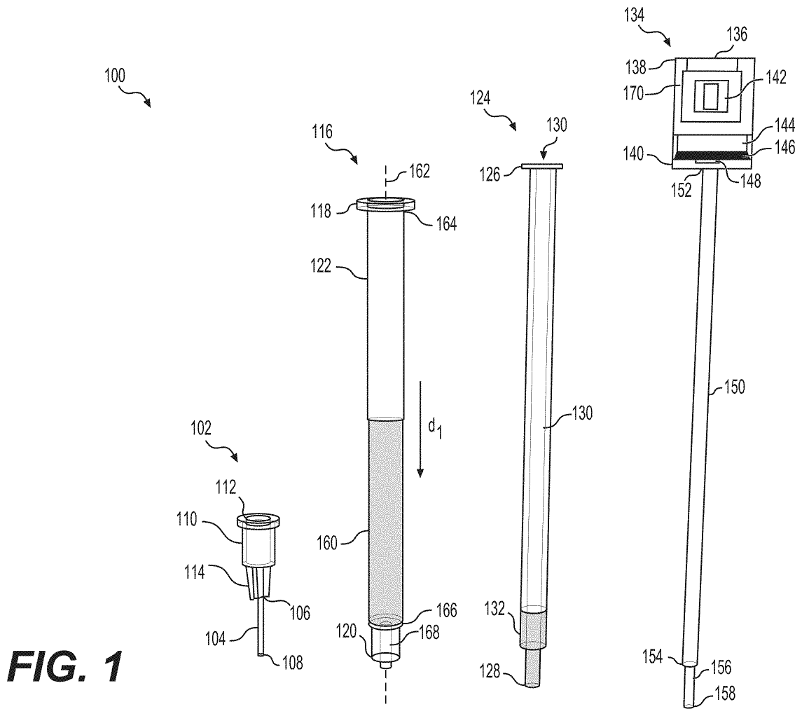

FIG. 1 illustrates component(s) of an example medical device 100 according to the present disclosure. In some examples, one or more components of the example medical device 100 may be manufactured utilizing existing manufacturing processes and/or technology. In some examples, one or more components of the example medical device 100 may require customized manufacturing and/or technology.

As illustrated in FIG. 1, an example medical device 100 may include a first component 102. The first component 102 may comprise a removeable needle and/or a head. The first component 102 may comprise a piercing member 104, which may comprise a needle (e.g., a 30-gauge needle or any needle of appropriate size). In some examples, the piercing member 104 may be hollow (e.g., it may have a central channel that extends from a proximal end 106 of the piercing member 104 to a distal end 108 thereof). In any of the examples described herein, the distal end 108 of the piercing member 104 may have an appropriate sharpness to pierce through one or more layers of tissue (e.g., cellular tissue, epidermal layers, and/or skin tissue) of the human eye. For example, the distal end 108 of the piercing member 104 may have a conical tip. The first component 102 may also comprise a base 110, which may include a proximal end 112 and a distal end 114. The base 110 may be composed of any material (e.g., plastics, polymers, metals, alloys, etc.) acceptable for use in a medical device. For instance, the base 110 may comprise a polymeric material and may be clear, translucent, or opaque. As illustrated in FIG. 1, the proximal end 106 of the piercing member 104 may be fixedly and/or fluidly attached to the distal end 114 of the base 110 and may define a fluid path extending from the first component 102 to the distal end 108 of the piercing member 104. The piercing member 104 may also be composed of a material standard in the medical field, such as a metal (e.g., steel, aluminum, alloy, copper, etc.).

Example medical device 100 may also include a second component 116. Second component 116 may comprise a syringe tube and may be removably attachable (e.g., such as via a fluid tight seal, snap fit, threads, and/or any other appropriate connection method) to base 110 of the first component 102. The second component 116 may have a proximal end 118 and a distal end 120. In some examples, the second component 116, may comprise a substantially cylindrical medical syringe tube having a central channel (e.g., a hollow center) 162 that extends from the proximal end 118 to the distal end 120 of the second component 116. In some examples, the proximal end 118 of the second component may comprise a flange coupled to an outer surface of the second component 116. In some examples, the second component comprises a cylindrical element 168 that enables the second component 116 to be removably attached to the third component 124 and the first component 102. The second component 116 may also comprise measurement marks along an outer and/or inner portion of the syringe tube. In some examples, the size of the piercing member 104 of the first component 102 and/or the base 110 of the first component 102 itself may be determined based on a size of the second component 116. For instance, if the second component 116 comprises a 1-millimeter syringe tube, the first component 102 may comprise a needle base and needle of a corresponding appropriate size. The second component 116 may also comprise a polymeric material and may be transparent or translucent. In some examples and unlike standard syringe tubes, the second component 116 may additionally include element 122. Element 122 may comprise a sleeve that is adjustable and slidably attached to an outer portion 160 of the second component 116. In some examples, element 122 may be composed of a light-blocking material (e.g., paper of appropriate thickness, plastics, polymers, etc.) and may slide from a first location 164 near the proximal end 118 of the second component 116 in a direction d1 towards a second location 166 near the distal end 120 of the second component 116. For instance, a user of the medical device 100 may draw an amount of fluid (e.g., a medicine and/or treatment) into the medical device 100. Once the desired amount of fluid is drawn into the medical device 100, element 122 on the second component 116 may slide in a direction d1 along a central longitudinal axis 162 from a first location 164 near the proximal end 118 of the second component 116 to a second location 166 near the distal end 120 of the second component 116 (e.g., slide down the second component 116). As illustrated, the central longitudinal axis 162 may extend substantially centrally through the second component from the proximal end 118 to the distal end 120. In some examples, element 122 may be used to cover a portion of the first component 102 (e.g., such as a portion of base 110). For instance, element 122 may comprise a sleeve that is slidably attached to the outer portion 160 of the second component 116 such that when element 122 is moved in a direction d1 from the first location 164 near the proximal end 118 of the second component 116 to the second location 166, element 122 may extend and/or stretch to cover a portion of the base 110. Accordingly, element 122 may be used to help block out and/or direct light through the medical device 100 (e.g., through the piercing member 104). By helping direct light through the medical device 100, element 122 may provide benefits to both users (e.g., by directing/focusing light radiation through the medical device 100), thereby improving visibility of the injection site.

Example medical device 100 may also include a third component 124. Third component 124 may comprise a syringe plunger with a proximal end 126 and a distal end 128. For example, a standard medical syringe plunger is manufactured to include a proximal end and a distal end, the distal end having a rubber boot attached at the distal end. However, unlike standard medical syringe plungers, the third component 124 may be manufactured to have a central channel 130 that extends along a central longitudinal axis from the proximal end 126 to the distal end 128 and allows radiation (e.g., light) to pass through. The third component 124 may also be manufactured to include element 132. Element 132 may comprise a cap that is attached at the distal end 128 of the third component 124. In some examples, the cap 132 is fluidly connected and/or fluidly sealed to the central channel 130 and may be configured to push items (e.g., fluid, medicine, etc.) through the tube 160. In some examples, the cap 132 may be removably attached to a distal end of the plunger, where the cap 132 comprises a third central channel fluidly connected to the second central channel of the plunger, the cap being configured to direct radiation emitted by the light source to exit the medical device via the distal end of the plunger. In some examples, the cap 132 may replace the rubber boot attached to standard medical syringe plungers. Cap 132 may comprise a polymeric material and may be clear, translucent, or opaque. Similar to the third component 124, cap 132 may also be hollow. The third component 124 may be composed of any material (e.g., plastics, polymers, metals, alloys, etc.). The third component 124 may be sized based on a size associated with the second component 116. For instance, where the second component 116 comprises a 1-millimeter syringe tube, the third component 116 may comprise a syringe plunger of an appropriate size, such that the third component 116 may be slidably disposed within the second component 116.

Example medical device 100 may also include a fourth component 134. In some examples, the fourth component 134 may comprise an optic assembly, such as a light emitting diode (LED) optic assembly. As illustrated in FIG. 1, the optic assembly 134 may include elements 136, 142, 144, 146, 148, 150, and 156. Element 136 may comprise a housing and may be made from any material (e.g., plastics, polymers, metals, alloys, etc.) acceptable for use with medical devices. Housing 136 may have a proximal end 138 and a distal end 140. Housing 136 may comprise one or more walls that may define an internal chamber and/or an interior space 170 configured to house one or more elements. In some examples, elements 142, 144, 146, and 148 may be fully and/or partially disposed within the interior space 170 of the housing 136. For instance, element 142 may comprise an input device (e.g., a button, a switch, a touch control, a capacitive input screen, etc.). In any of the examples described herein, the input device may be connected to and/or supported by the housing 136. Element 144 may comprise a battery and/or a power source (e.g., 3.3V, any suitable size of battery, or any acceptable power source), which may be communicatively, electronically, or operably connected to input device 142 and disposed within housing 136. For instance, power source 144 may be electronically connected to input device 142 via a shunt bus (not shown). Element 146 may comprise a circuit board that may be communicatively, operably, or electronically coupled to power source 144 and/or to the input device, 142. In any of the examples described herein, the element 146 (i.e., the circuit board) may be disposed within housing 136. In some examples, the circuit board may comprise one or more processor(s), microprocessor(s), controller(s), driver(s), and/or other control components. Element 148 may comprise a light source (e.g., one or more LEDs or other sources of light) disposed within housing 136, that may be mounted to, communicatively, operably, or electronically connected to circuit board 146. In some examples, light source 148 may emit radiation (e.g., light) as a visual feedback, in the form of one or more color(s), pattern(s), brightness(es), intensities, etc. In some examples, the light source 148 selectively emits radiation in response to signals received from a controller of the circuit board 146. Such signals are generated by controller based on input received from input device 142.

As illustrated in FIG. 1, the fourth component 134 may also include element 150. Element 150 may comprise a tube (such as a fiber optic light pipe, an aluminum tube, a polymeric tube, etc.) with a central channel extending from a proximal end 152 and a distal end 154. Tube 150 may be electronically and/or operably connected to light source 148. The proximal end 152 of tube 150 may be attached at the distal end 140 of housing 136. For instance, tube 150 and light source 148 may be attached through various manners and/or using various techniques (e.g., brazed, soldered, welded, glued (e.g., with a conductive glue), heated together, connected with an adhesive (e.g., a conductive adhesive), or otherwise joined together. In some examples, tube 150 encase element 156, such that element 156 is disposed within tube 150. Element 156 may comprise a light pipe and/or fiber optic tube comprising a polymeric material and have a proximal end 152 and a distal end 158. In some examples, light pipe 156 may comprise a light pipe tube (e.g., a fiber optic tube) that encases a polymeric tube. In some examples, tube 150 may comprise a polymeric tube that encases a polymeric fiber optic tube. Tube 150, including a light pipe 156, may also be of a substantially same length as the third component 124. Light pipe 156 may be sealed at the distal end 158. In some examples, the distal end 158 of light pipe 156 may be sealed in a curved manner (e.g., convex, etc.), such that the distal end may be configured to operate as an optic, such that radiation emitted from light source 148 and directed from the proximal end 152 of tube 150 to the distal end 158 of light pipe 156 may be concentrated and/or diffused.

In some examples, the components 102, 116, 124, and 134 may comprise a reusable medical device 100 (e.g., such as a reusable syringe). In this example, tube 150 of the fourth component 134 may comprise a hollow tube made from a conductive material (e.g., aluminum, alloy, or other such material appropriate for use), that encases light pipe 156, in order to enable the fourth component 134 to provide longevity to users of medical device 100 and withstand wear from being used multiple times (e.g., such as being sanitized after use and/or not damaged from the cleaning and/or sanitization process and/or chemicals). In some examples, the third component 124 may include one or more elements of the fourth component 134. For instance, where medical device 100 represents a disposable medical device (e.g., such as a one-time use syringe), one or more of elements 136, 142, 144, 146, 148, 150, and 156 may be integrated into the third component 124. In this example, tube 150 may comprise a polymeric material (e.g., plastic, polymer, etc.) that encases the light pipe 156.

In some examples, one or more of the components 102, 116, 124, and 134 of medical device 100 may be reusable. For example, the fourth component 134 may comprise elements 136, 142, 144, 146, 148, 150, and 156, and may be reusable between medical devices 100. In this example, the fourth component may be used as part of a first medical device 100 to deliver a first treatment. Components 102, 116, and/or 124 of the first medical device 100 may be disposed of after delivering the first treatment. The fourth component 134 may be reused (e.g., after any needed and/or required sanitization, cleaning, etc.), as the fourth component 134 of a second medical device 100 to deliver a second treatment, and so on. In some examples, the third component 124 and the fourth component 134 may be integrated into a single, reusable component. For instance, one or more of elements 136, 142, 144, 146, 148, 150, and 156 may be integrated into the third component 124 to create a reusable third component 124, which may be used as part of a first medical device 100 to deliver a first treatment. In this example, the reusable third component 124, after any needed and/or required sanitization, cleaning, etc., may be used as part of a second medical device 100 to deliver a second treatment, and so on.

In some instances, a conductive filler (e.g., a metal) may be applied between one or more of elements 136, 142, 144, 146, 148, 150, and 156 of the fourth component 134 to make a connection, while in other instances the elements 136, 142, 144, 146, 148, 150, and 156 may be directly attached to each other. The elements 136, 142, 144, 146, 148, 150, and 156 of the fourth component 134 may be directly or indirectly connected. The terms “connected” or “electrically connected” may refer to elements 136, 142, 144, 146, 148, 150, and 156 directly contacting each other or indirectly contacting each other. In some examples, elements 136, 142, 144, 146, 148, 150, and 156 may directly contact each other or indirectly contact each other through a conductive filler.

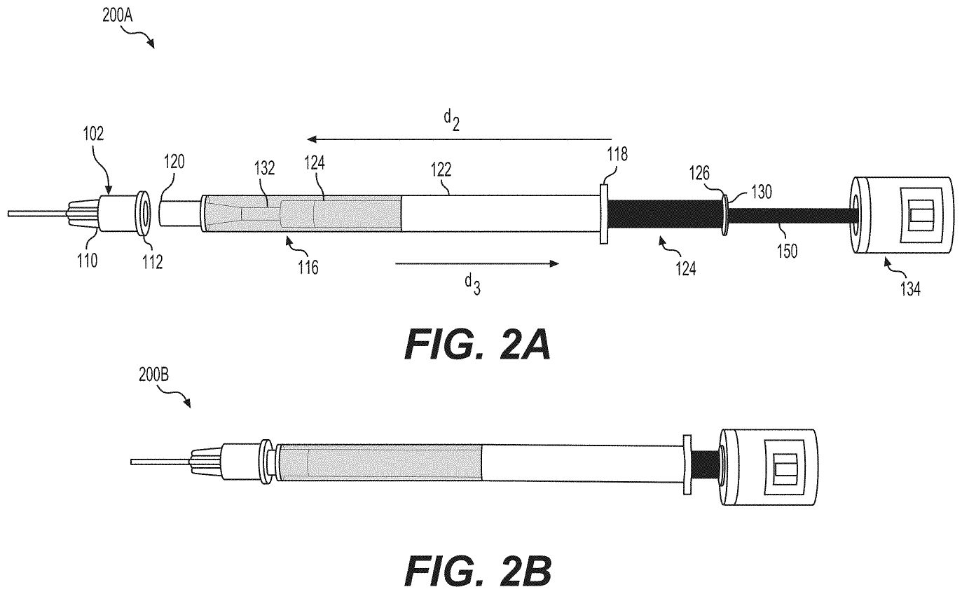

FIG. 2A illustrates a partially exploded view 200A of the example medical device 100 shown in FIG. 1. FIG. 2B illustrates an example view 200B of an assembled example medical device 100, as described in FIGS. 1 and 2A. As noted above, medical device 100 may include components 102, 116, 124, and 134. As illustrated in FIG. 2A, the first component 102 may be removably attached to the second component 116. For instance, the proximal end 112 of the base 110 may be attached to the distal end 120 of the second component 116. The first component 102 may have the piercing member 104 fixedly and/or fluidly attached to the distal end 114 of the base 110, such that the second component 116 forms a substantially fluid-tight seal with base 110. The distal end 120 of the second component 116 may be inserted in a fist direction d2 into the proximal end 112 of the base 110 of the first component 102, such that the distal end 120 of the second component 116 is encased by (e.g., disposed within) the base 110 of the first component 102 and may removably attach the first component 102 to the second component 116. Additionally, the central channel of the piercing member 104 may be fluidly attached to the central channel of the second component 116. As illustrated in FIG. 2A, the second component 116 may comprise element 122, which may comprise a sleeve that is adjustable and slidably attached to outer portion of the second component 116. In some examples, element 122 may be composed of a light-blocking material (e.g., paper of appropriate thickness, plastics, polymers, etc.) and may slide in a first direction d2 from a first location near the proximal end 118 of the second component 116 towards a second location near the distal end 120 of the second component 116.

In some examples, the distal end 128 (not shown) of the third component 124 is inserted in a first direction d2 into the central channel of the second component 116, such that the second component 116 encases the third component 124. The distal end 128 (not shown) of the third component 124 may be inserted into the proximal end 118 of the second component 116 and move in a first direction d2 towards the distal end 120 of the second component 116, until the distal end 128 of the third component 124 is substantially parallel with the distal end 120 of the second component 116. In some examples, the third component 124 is rotatable, pivotable, slidable, and/or otherwise movable relative to the first component 102 and the second component 116.

In some examples, the distal end 158 (not shown) of the fourth component 134 is inserted into the central channel 130 at the proximal end 126 of the third component 124 and moved in a first direction d2 towards the distal end 128 of the third component 124, such that the third component 124 may substantially encase the tube 150 and light pipe 156 of the fourth component 134. The fourth component 134 may be inserted in a first direction d2 towards the distal end 128 of the third component 124, at least until a portion of light pipe 156 is encased by cap 132 of the third component 124 and/or the distal end 158 of the fourth component 134 is substantially parallel with the distal end 128 of the third component 124. In some examples, the fourth component 134 is attached to the third component 124, such that the fourth component 134 is rotatable, pivotable, slidable, and/or otherwise movable relative to the first component 102 and the second component 116, such as when the third component 124 is moved. For example, when a user draws fluid (e.g., medicine, treatment, etc.) into the medical device 100, the user may slide the distal end 128 of the third component 124 in a second direction d2, towards the proximal end 118 of the second component 116 until the medical device 100 contains a desired amount of fluid within the second component 116. As the third component 124 is sliding in the second direction d2, the fourth component 134 is attached to the third component 124, such that the fourth component 134 slides in the same direction d2, at the same time, and travel a substantially same distance as the third component 124 relative to the first component 102 and the second component 116. Accordingly, when input device 142 receives an input (e.g., pressure, indication of selection, etc.) input device 142 may be configured to selectively cause power source 144 to direct a current to light source 148, thereby causing light source 148 to emit radiation in response to the input. The radiation may then be directed from the proximal end 152 of tube 150 to the distal end 158 of light pipe 156, and through the distal end 128, of cap 132, the distal end 120 of the second component 116, and the distal end 114 of base 110, such that the radiation is directed through the distal end 108 of piercing member 104 of the medical device 100. Accordingly, when assembled, the medical device 100 may appear as illustrated in FIG. 2B.

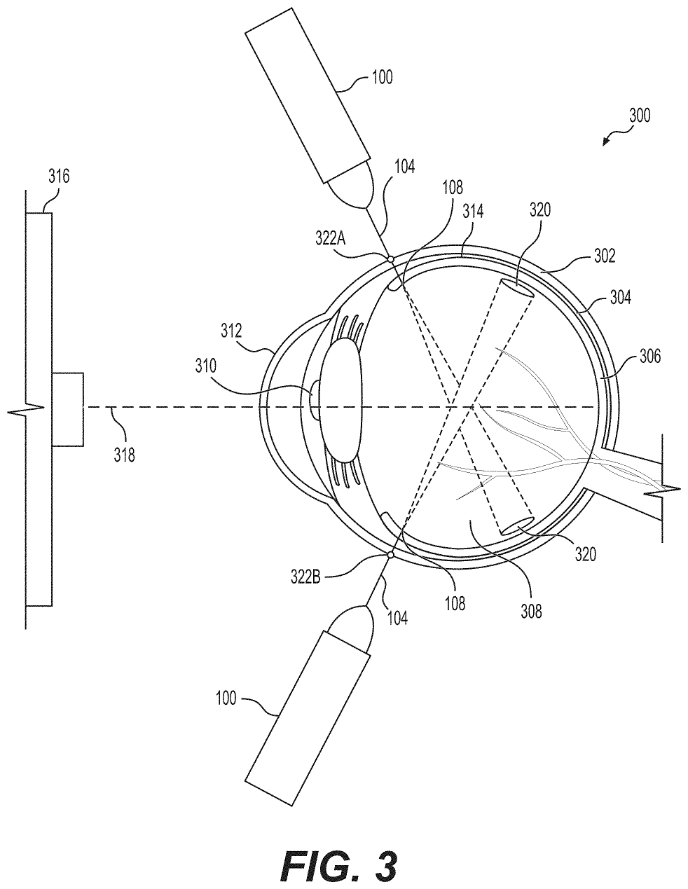

FIG. 3 illustrates an example cross sectional view of a human eye 300. As noted above, the eye 300 may include various layers of tissue 302, 304, 306. As noted above, a first layer of tissue 302 may comprise the sclera, a second layer of tissue 304 may comprise the choroid, and a third layer of tissue 306 may comprise the retina. As illustrated in FIG. 3, the eye 300 may also include the vitreous 308, the pupil 310, and the cornea 312. As described above and illustrated in FIG. 3, a pocket and/or gap 314 may exist between the second layer of tissue 304 and the third layer of tissue 306. When administering medicine to the eye 300 of a patient, determining when the medical device 100 has reached a proper depth (e.g., such as the pocket and/or gap 314) is important to ensure the patient's treatment is effective.

In some examples, a user (e.g., physician, primary care physician, etc.)(not shown) may use medical device 100 to monitor depth when providing a treatment to a patient. For example, a device 316 (e.g., ophthalmoscope, etc.) may be used by a user to look into a patient's eye 300 through the patient's pupil 310. The device 316 may provide the user with a line of site 318 within the eye 300. In some examples, medical device 100, may be inserted into the eye 300 at one or more insertion point(s) 322. For example, as illustrated in FIG. 3, the distal end 108 of the piercing member 104 may be inserted into the eye 300 at insertion point 322A or 322B. Insertion point 322A or 322B may correspond to respective locations that are a certain distance from the cornea 312. As noted above, the pocket and/or gap 314 between the choroid 304 and retina 306 may vary depending on how close to the cornea 312 the insertion point 322A or 322B may be. For instance, the closer to the cornea 312 insertion point 322A or 322B is, the smaller the pocket and/or gap 314 may be. Accordingly, for purposes of this example, insertion point 322A or 322B may comprise respective locations that are respective particular distances (e.g., such as 7-8 millimeters) away from the cornea 312. Such respective locations and/or distances may be associated with a targeted/optimal pocket and/or gap 314 for the delivery of medicine or other treatments within the eye 300.

When the distal end 108 of the piercing member 104 of the medical device 100 has pierced through the retina 306, the medical device 100 may provide visual feedback 320 to the user (e.g., via the line of site 318). For instance, the medical device 100 may enable the user to visually identify the radiation (e.g., visual feedback 320) that is impinging on a component of the eye 300. The visual feedback 320 (e.g., radiation emitted from light source 148 and directed through the distal end 108 of the piercing member 104) may be in the form of one or more color(s), a brightness, a pattern, a light spot, etc., that is visible to the user of the device 316, via the user's line of site 318. In some examples, the visual feedback 320 may appear on any portion of the back of the eye 300. In some examples, the location of the visual feedback 320 may be affected by one or more factors (e.g., angle of the medical device 100, location of insertion point 322A or 322B, distance from cornea 312, among other things).

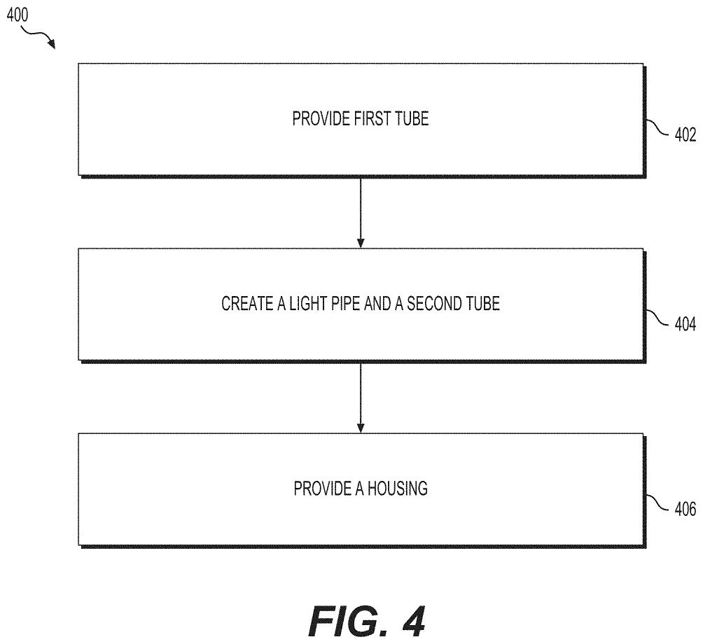

FIG. 4 illustrates an example method 400 associated with the example medical device 100 described in FIG. 1 above. In some examples, method 400 may correspond to manufacturing and/or assembling the medical device 100 described in FIG. 1.

The example method 400 is illustrated as a logical flow graph, each operation of which represents a sequence of operations that may be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be combined in any order and/or in parallel to implement the processes.

At step 402, the method 400 comprises providing a first tube. In some examples, the first tube comprises a proximal end and a distal end. The first tube may further comprise a first central channel extending from the proximal end to the distal end. In some examples, the first tube may comprise tube 150 described above.

As noted above, tube 150 may comprise a tube (such as a fiber optic light pipe, an aluminum tube, a polymeric tube, etc.) with a central channel extending from a proximal end 152 and a distal end 154. Tube 150 may be electronically and/or operably connected to light source 148. The proximal end 152 of tube 150 may be attached at the distal end 140 of housing 136. For instance, tube 150 and light source 148 may be attached through various manners and/or using various techniques (e.g., brazed, soldered, welded, glued (e.g., with a conductive glue), heated together, connected with an adhesive (e.g., a conductive adhesive), or otherwise joined together. In some examples, tube 150 encase element 156, such that element 156 is disposed within tube 150.

At step 404, the method 400 comprises creating a light pipe tube and a second tube. In some examples, the light pipe tube is disposed at least partly within a second central channel of the second tube to create a light pipe 156, the light pipe being disposed at least partly within the first central channel of the first tube 150. In some examples, the light pipe tube and the second tube may be created using one or more injection mold(s). In some examples, the light pipe 156 may be configured to receive radiation emitted by a light source and direct the radiation through the distal end 158 of the first tube 150. As described above, light pipe 156 may comprise a light pipe and/or fiber optic tube comprising a polymeric material and have a proximal end 152 and a distal end 158. In some examples, light pipe 156 may comprise a light pipe tube (e.g., a fiber optic tube) that encases a polymeric tube. In some examples, tube 150 may comprise a polymeric tube that encases a polymeric fiber optic tube. Tube 150, including a light pipe 156, may also be of a substantially same length as the third component 124. Light pipe 156 may be sealed at the distal end 158. In some examples, the distal end 158 of light pipe 156 may be sealed in a curved manner (e.g., convex, etc.), such that the distal end 158 may be configured to operate as an optic, such that radiation emitted from light source 148 and directed from the proximal end 152 of tube 150 to the distal end 158 of light pipe 156 may be concentrated and/or diffused.

At step 406, the method 400 comprises providing a housing 136. In some examples, the housing 136 is attached to the proximal end 152 of the first tube 150 and operably connected to the light pipe 156 to create an optic assembly 134. In some examples, the housing 134 may comprise a power source 144 disposed with the housing 134 and connected to an input device 142, a circuit board 146 disposed within the housing 136 and connected to the power source 144, and a light source 148 disposed within the housing 136 and connected to the circuit board 146 and operably connected to the light pipe 156.

As described above, the housing 136 may be made from any material (e.g., plastics, polymers, metals, alloys, etc.) acceptable for use with medical devices. Housing 136 may have a proximal end 138 and a distal end 140. Housing 136 may comprise one or more walls that may define an internal chamber and/or space configured to house one or more elements. In some examples, elements 142, 144, 146, and 148 may be fully and/or partially disposed within housing 136. For instance, element 142 may comprise an input device (e.g., a button, a switch, a touch control, etc.). Element 144 may comprise a battery and/or a power source (e.g., 3.3V, any suitable size of battery, or any acceptable power source), which may be communicatively, electronically, or operably connected to input device 142 and disposed within housing 136. For instance, power source 144 may be electronically connected to input device 142 via a shunt bus (not shown). Element 146 may comprise a circuit board that may be communicatively, operably, or electronically coupled to power source 144 and disposed within housing 136. Element 148 may comprise a light source (e.g., one or more LEDs or other sources of light) disposed within housing 136, that may be mounted to, communicatively, operably, or electronically connected to circuit board 146. In some examples, light source 148 may emit radiation (e.g., light) as a visual feedback, in the form of one or more color(s), pattern(s), brightness(es), intensities, etc.

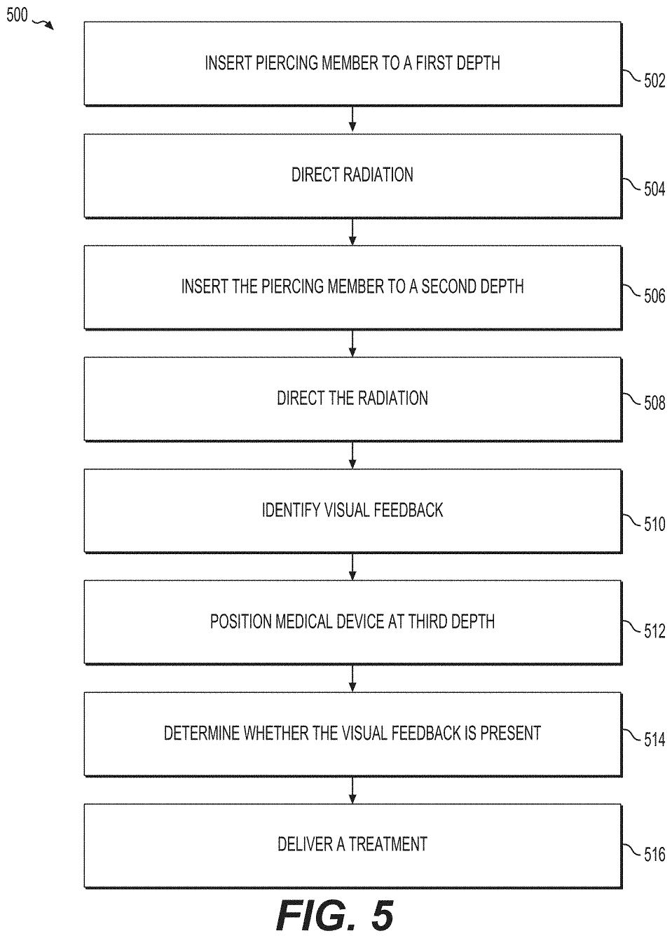

FIG. 5 illustrates another example method 500 associated with the example medical device described in FIG. 1 above. For instance, method 500 may correspond a method of using the example medical device 100 described in FIG. 1 above. For instance, as described above, the example medical device 100 may direct radiation emitted from a light source 148, through the distal end 108 of a piercing member 104.

The example method 500 is illustrated as a logical flow graph, each operation of which represents a sequence of operations that may be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be combined in any order and/or in parallel to implement the processes.

At step 502, the method 500 comprises inserting a piercing member. In some examples, the piercing member comprises a piercing member 104 of a medical device 100, and may be inserted through a sclera 302 of an eye 300, to a first depth within the eye 300. As described above, the medical device 100 may include components 102, 116, 124, and 134. For instance, the medical device 100 may comprise (i) a head 102 having a base 110 and a piercing member 104 fluidly attached to the base 110, (ii) a syringe tube 116, the syringe tube being substantially cylindrical and having a proximal end 118, a distal end 120, the distal end 120 of the syringe tube 116 being removably attached to the base 110, (iii) a syringe plunger 124 slidably disposed within a first central channel of the syringe tube 116, the syringe plunger defining a second central channel 130, and (iv) an optic assembly 134 having a light source 142 and a light pipe 156, the light pipe 156 being disposed at least partly within the second central channel 130. As described above, when medical device 100 is inserted into an eye 300, the piercing member 104 may pierce through one or more layers of tissue 302, 304, 306, within the eye 300. Accordingly, in some examples, the first depth within the eye 300 may correspond to a depth between the sclera 302 and the choroid 304. In other examples, the first depth may correspond to a depth between the choroid 304 and the retina 306.

At step 504, the method 500 comprises directing radiation. In some examples, the radiation is directed, through the piercing member 104, to impinge upon a surface of a retina 306 of the eye 300 at a first location. For instance, as noted above, the first depth within the eye 300 may correspond to a depth between the between the sclera 302 and the choroid 304 and/or the choroid 304 and the retina 306. When input is provided to input device 142 of the medical device 100, the input device 142 may direct power source 144 to direct a current to cause light source 148 to emit radiation. The radiation is directed through the piercing member 104, such that a user may determine if a visual feedback 320 is present at a particular location within the eye 300, such as within a user's line of site 318. In this example, the piercing member 104 has not yet pierced through the retina 306, such that no visual feedback 320 is present. Accordingly, the method continues to step 506.

At step 506, the method 500 comprises inserting the piercing member 104. In some examples, the piercing member 104 of the medical device is inserted, through the retina 306 of the eye 300, to a second depth within the eye 300. As indicated above, once the piercing member 104 has pierced through the retina 306, visual feedback 320 may be visible within the eye 300. The second depth may correspond to the piercing member 104 entering the vitreous 308.

At step 508, the method 500 comprises directing the radiation. In some examples, the radiation is directed through the piercing member 104, to impinge upon a surface of an innermost area of the eye 300 at a second location. As described above, the innermost area of the eye 300 may comprise the vitreous 308 (e.g., vitreous body) of the eye and the second location may comprise any location along the back of a patient's eye 300, such as a location within the user's line of site 318.

At step 510, the method 500 comprises, identifying visual feedback 320. In some examples, the visual feedback 320 is identified, on the surface of the innermost area of the eye 300, at the second location, the visual feedback 320 being associated with the second depth of the medical device 100. As noted above, the visual feedback 320 may be visible to a user along any point within the back of the eye 300. The visual feedback 320 may comprise any concentration, color, shape, pattern, etc. of radiation that is emitted from light source 148.

At step 512, the method 500 comprises positioning the medical device 100 at a third depth. In some examples, the medical device 100 is caused, based on identifying the visual feedback 520, the medical device 100 to be positioned at the third depth within the eye 300. For instance, once the visual feedback 320 is identified, the medical device 100 may be determined to be too deep within the eye 300 to provide effective treatment. Accordingly, the medical device 100 may be positioned at a third depth within the eye 100. In some examples, the third depth may comprise a depth that is shallower than the second depth (e.g., the piercing member 104 of the medical device 100 is withdrawn and/or pulled out of the eye 300 a particular distance).

At step 514, the method 500 comprises determining, whether the visual feedback 320 is present. For instance, the determining may be based on the third depth. In some examples, the visual feedback 320 is determined to be present or not present at the second location on the surface of the innermost area of the eye 300. For instance, as indicated above, the third depth may comprise a depth that is shallower than the second depth. In this example, the third depth may comprise a depth between the retina 306 and the choroid 304, such as the pocket and/or gap 314. Accordingly, once the piercing member 104 is no longer piercing the retina 306, the visual feedback 320 may no longer be present at the second location on the innermost area of the eye 300. In some examples, the lack of visual feedback 320 being present may indicate to a user of the medical device 100 that the piercing member 104 is positioned at a targeted depth (e.g., pocket and/or gap 314) within the eye 300 for providing a treatment to a patient.

At 516, the method 500 comprises delivering a treatment. For instance, the treatment is delivered based on determining that a lack of visual feedback 320 is present on the innermost area of the eye 300, causing the medical device 100 to deliver the treatment within the eye 300. In some examples and as described above, the treatment may be delivered at the third depth, such as when it is determined that the visual feedback 320 is no longer present on the innermost area of the eye 300 and/or at the second location, when the medical device 100 is positioned at the third depth. In other examples, the visual feedback 320 may still be present at the second location on the innermost area of the eye 300 at the third depth. In this example, based on determining that visual feedback 320 is still present, the medical device 100 may be positioned at a fourth depth within the eye 300. As described above, the fourth depth may be a depth that is shallower than the third depth. In this example, the user may determine, based on the fourth depth, that there is the lack of visual feedback 320 present on the innermost area of the eye 300 at the second location. Thus, the user may determine that the fourth depth corresponds to a targeted depth (e.g., pocket and/or gap 314) to deliver the treatment. Upon this determination, the user may cause the medical device 100 to deliver the treatment within the eye 300 at the fourth depth.

Based at least on the description herein, it is understood that the devices and methods of the present disclosure may be used to deliver medication via subretinal injection. For example, components described herein may be configured to provide an illuminated syringe plunger, such that a stream of light is illuminated through a needle attached to the syringe, such that a dot of light is shown on the back of a patient's retina when the retina has been pierced. As a result, the devices and methods described herein may assist a user with administering medication to a patient at a depth that is specific to the patient, thereby improving effectiveness and accuracy of the treatment. This may also reduce the number of invasive procedures needed and streamline workflow for providing treatments for primary care physicians and others, thereby reduce the cost of treatments.

The foregoing is merely illustrative of the principles of this disclosure and various modifications can be made by those skilled in the art without departing from the scope of this disclosure. The above described examples are presented for purposes of illustration and not of limitation. The present disclosure also can take many forms other than those explicitly described herein. Accordingly, it is emphasized that this disclosure is not limited to the explicitly disclosed methods, systems, devices, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices, and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

What is claimed is:

1. A medical device, comprising:
a head having a base and a piercing member fluidly connected to the base;
a tube having a proximal end, a distal end, a first central channel extending from the proximal end to distal end, and a longitudinal axis extending substantially centrally through the first central channel, the distal end of the tube being removably attached to the base;
a plunger slidably disposed at least partly within the first central channel of the tube, the plunger defining a second central channel, and the longitudinal axis extending substantially centrally through the second central channel; and
an optic assembly having a light source and a light pipe, the light pipe being disposed at least partly within the second central channel and being configured to:
receive radiation emitted by the light source; and
direct the radiation through the piercing member.

2. The medical device of claim 1, wherein the tube is substantially cylindrical, and when the tube is removably attached to the base:
the tube forms a substantially fluid-tight seal with the base, and
the first central channel of the tube is fluidly connected to a third central channel of the piercing member.

3. The medical device of claim 1, wherein the light source comprises a light emitting diode (LED).

4. The medical device of claim 3, wherein the optic assembly further includes a housing and a circuit board at least partly disposed within the housing, the LED being mounted to the circuit board.

5. The medical device of claim 4, wherein the optic assembly further includes a power source operably connected to the LED and at least partly disposed within the housing.

6. The medical device of claim 4, further comprising a light pipe tube connected to the housing, disposed at least partly within the second central channel of the plunger, and defining a third central channel, the longitudinal axis extending substantially centrally through the third central channel, and the light pipe being disposed at least partly within the third central channel.

7. The medical device of claim 1, wherein the tube further comprises a sleeve slidably moveable along an outer surface of the tube, the sleeve being configured to at least partially block the radiation emitted by the light source from passing through the light pipe.

8. The medical device of claim 1, wherein the plunger further comprises a cap removably attached to a distal end of the plunger, the cap having a third central channel fluidly connected to the second central channel of the plunger, the cap being configured to direct radiation emitted by the light source to exit the medical device via the distal end of the plunger.

9. The medical device of claim 1, wherein the optic assembly further comprises an input device at least partly disposed within a housing and operably connected to a power source, the input device being configured to selectively cause the power source to direct current to the light source in response to receiving an input.

10. The medical device of claim 1, wherein the light pipe comprises at least one of a conductive material or a polymeric material.

11. A method, comprising:
providing a first tube comprising a proximal end, a distal end, a first central channel extending from the proximal end to the distal end, and a longitudinal axis extending substantially centrally through the first central channel;
providing a light pipe comprising a light pipe tube and a second tube, wherein the light pipe tube is disposed at least partly within a second central channel of the second tube;
disposing the light pipe at least partly within the first central channel of the first tube;
connecting a housing to the proximal end of the first tube and operably connected to the light pipe to create an optic assembly, the housing defining an interior space and supporting an input device; and
disposing a power source, a light source, and a circuit board at least partly within the interior space of the housing,
the circuit board being operably connected to the power source, the light source, and the input device,
the light source being operable to selectively direct radiation to the light pipe based on an input received via the input device, and
the light pipe being configured to direct the radiation to exit the first tube via the distal end of the first tube.

12. The method of claim 11, wherein the first tube is composed of a conductive material and the second tube is composed of a polymeric material.

13. The method of claim 11, wherein the first tube and the second tube are composed of a polymeric material.

14. The method of claim 11, further comprising:
providing a third tube having a proximal end and a distal end, the third tube having a third central channel extending from the proximal end of the third tube to the distal end of the third tube;
removably attaching a head to the distal end of the third tube; and
disposing a plunger within the third central channel, the plunger having a fourth central channel and being slidably moveable within the third central channel.

15. The method of claim 14, wherein the light pipe is disposed at least partly within the third central channel of a plunger.

16. A method, comprising:
inserting a piercing member of a medical device, through a sclera of an eye, to a first depth within the eye, the medical device comprising:
a head having a base and the piercing member fluidly attached to the base;
a tube having a proximal end, a distal end, a first central channel extending from the proximal end to distal end, and a longitudinal axis extending substantially centrally through the first central channel, the distal end of the tube being removably attached to the base;
a plunger slidably disposed at least partly within the first central channel of the tube, the plunger defining a second central channel, and the longitudinal axis extending substantially centrally through the second central channel; and
an optic assembly having a light source and a light pipe, the light pipe being disposed at least partly within the second central channel;
directing first radiation, through the piercing member, to impinge upon a retina of the eye at a first location;
inserting the piercing member of the medical device, through the retina of the eye, to a second depth within the eye;
while the piercing member is disposed at the second depth:
directing second radiation through the piercing member to impinge upon an innermost area of the eye at a second location, and
visually identifying the second radiation impinging upon the innermost area of the eye at the second location;
based on visually identifying the second radiation, causing the piercing member to be disposed at a third depth within the eye;
while the piercing member is disposed at the third depth:
determining that the second radiation cannot be visually identified as impinging upon the innermost area of the eye at the second location, and
based on determining that the second radiation cannot be visually identified as impinging upon the innermost area of the eye, delivering a treatment within the eye via the piercing member.

17. The method of claim 16, wherein the innermost area of the eye comprises a vitreous body of the eye.

18. The method of claim 16, wherein the third depth is less than the second depth and comprises a targeted depth to deliver the treatment.

19. The method of claim 16, wherein the first radiation and the second radiation is emitted by the light source of the medical device, the first radiation and the second radiation being emitted as one or more colors, brightness, patterns, or intensities.

20. The method of claim 16, further comprising:
determining, based on the third depth, that the third radiation is present on the innermost area of the eye at the second location;
causing, based on the determining, the medical device to be positioned at a fourth depth within the eye;
determining, based on the fourth depth, that there is the lack of the third radiation present on the innermost area of the eye at the second location;
determining that the fourth depth corresponds to a targeted depth to deliver the treatment; and
causing the medical device to deliver the treatment within the eye at the fourth depth.
